(19) United States
(12) Patent Application Publication
(10) Pub. No.: US 2026/0364322 A1
(43) Pub. Date: Nov. 19, 2026

(54) INGESTIBLE ELECTRONICALLY TRIGGERED DRUG DELIVERY SYSTEM

(71) Applicant: CELERO SYSTEMS, INC., Lincoln, MA (US)

(72) Inventors: Benjamin David Pless, Lincoln, MA (US); Daniel Bacher, Lincoln, MA (US); Brett M. Wingeier, Lincoln, MA (US)

(21) Appl. No.: 19/139,554

(22) PCT Filed: Dec. 20, 2023
(86) PCT No.: PCT/US2023/085026

§ 371 Date: Jun. 16, 2025

Related U.S. Application Data
(60) Provisional application No. 63/433,856, filed on Dec. 20, 2022.
(60) Provisional application No. 63/451,718, filed on Mar. 13, 2023.

(51) Int. Cl. A61M 31/00; A61B 5/0205

(52) U.S. Cl. CPC A61M 31/002; A61B 5/02055

(57) ABSTRACT

An ingestible capsule configured to release a therapeutic agent(s) (26) via an electrothermal-chemical reaction is provided. The capsule contains a chamber with an inflatable microbag (20) and a therapeutic agent (26). The interior of the microbag (20) includes a heating filament and a chemical substance in communication with the heating filament such that a chemical reaction is initiated when the chemical substance is heated by the heating filament to produce a gas. The ingestible capsule also has capsule electronics that includes a sensor and controller. The controller can process sensed data from the sensor and electrically trigger the heating filament to generate heat sufficient to initiate the chemical reaction to produce the gas to inflate the respective microbag (20) to a sufficient size and configuration to eject the therapeutic agent (26) out of capsule when a physiological event indicative of a medical condition is detected.

### CROSS-REFERENCE TO RELATED APPLICATIONS

The present application claims priority to U.S. Provisional Application No. 63/433,856 filed on Dec. 20, 2022 and U.S. Provisional Application No. 63/451,718 filed on Mar. 13, 2023 both of which are incorporated by reference in their entirety.

### TECHNICAL FIELD

The present disclosure relates to an ingestible capsule configured to release a therapeutic agent(s) via an electrothermal-chemical reaction.

### BACKGROUND

Ingestible capsules have been described for delivering a therapeutic agent via a thermochemical reaction. For example, CN-100534544 describes a remotely controlled slow-releasing electronic capsule for releasing medicine in the digestive tract that is composed of a casing, power supply unit, control circuit, piston, medicine-releasing channel, and a driver having a propulsion device. The propulsion device consists of an igniter, propellant cabin, nozzle and propellant for applying a propelling force to the piston to open the medicine-releasing channel for slowly releasing the medicine. CN-113769250 describes a drug delivery capsule that has a shell, a soft bag arranged in the shell, and a drug delivery channel that communicates with the soft bag and the outside of the shell. The soft bag is arranged in a medicine bin and an equipment cavity is internally provided with a gas bin for filling pressurized gas. A connecting channel for communicating the medicine bin with the gas bin is arranged on a partition plate. The drug delivery capsule is also provided with a valve body assembly for controlling the opening and closing of the connection channel. Pressurized gas pre-stored in the equipment cavity is controlled to enter the medicine bin, so that the pressurized gas occupies the space of the medicine bin. The soft bag is extruded and compressed and the medicine flows out of the soft bag. CN-108686292 describes a medicine releasing capsule that includes a control cabinet, a first drug release cabin, and a second drug release cabin. The control cabinet includes a wireless receiving module, minicell and two microprocessors. After the medicine releasing capsule reaches the gastrointestinal tract and when the control cabinet receives an external trigger signal for the first time, the first drug release cabin discharges drug. When the control cabinet receives an external trigger signal for the second time, the second drug release cabin discharges drug.

However, none of these capsules provide a mechanism by which the timing of drug release can be controlled. It is desirable for drug to be released from the capsule upon a determination that the onset of the patient's medical condition has occurred.

### SUMMARY

In an aspect, an ingestible capsule to improve a medical condition in a patient suffering therefrom is provided that includes a first portion and a second portion. The first portion can include a chamber with a microbag and therapeutic agent therein. The microbag can have an outer surface and comprise a heating filament and a chemical substance in communication with the heating filament such that a chemical reaction is initiated when the chemical substance is heated by the heating filament to produce a gas. The therapeutic agent can be disposed outside of the microbag and a lid can be attached to the chamber and can be configured to releasably retain the therapeutic agent. The second portion of the ingestible capsule can include capsule electronics comprising a sensor and controller. The sensor can be configured to detect a parameter and generate a sensor signal in response to the detection. The controller can be operably coupled to the chamber and programmed to, receive the sensor signal, identify or estimate a biomarker relevant to the medical condition based on the sensor signal, determine whether a physiological event characteristic of the medical condition has occurred based on the identification or estimation, and electrically trigger the heating filament to generate heat sufficient to initiate the chemical reaction to produce the gas to inflate the microbag to a sufficient size and configuration to dislodge the lid and thereby eject the therapeutic agent out of the chamber into the gastrointestinal tract upon a determination that the event has occurred.

In another aspect, an ingestible capsule is provided that has a first portion, a second portion, and a longitudinal axis extending therethrough. The first portion can include a plurality of chambers radially arranged about the longitudinal axis of the ingestible capsule. Each of the plurality of chambers can comprise a microbag and a therapeutic agent. The microbag can have an outer surface and comprise a heating filament and a chemical substance in communication with the heating filament such that a chemical reaction is initiated when the chemical substance is heated by the heating filament to produce a gas. The therapeutic agent can be disposed outside of the microbag. The ingestible capsule can also include a plurality of lids, each attached to a respective one of the plurality of chambers and configured to releasably retain the therapeutic agent. The second portion of the ingestible capsule can include capsule electronics comprising a sensor and a controller. The sensor can be configured to detect a parameter and generate a sensor signal in response to the detection. The controller can be operably coupled to each of the plurality of chambers and programmed to receive the sensor signal, identify or estimate a biomarker relevant to the medical condition based on the sensor signal, determine whether a physiological event characteristic of the medical condition has occurred based on the identification or estimation, and electrically trigger the heating filament to generate heat sufficient to initiate the chemical reaction to produce the gas to inflate the respective microbag to a sufficient size and configuration to dislodge the respective lid and thereby eject the therapeutic agent out of the respective one of the plurality of chambers into the gastrointestinal tract upon a determination that the event has occurred.

### BRIEF DESCRIPTION OF THE DRAWINGS

FIG. 1 is a perspective view of an ingestible capsule according to an aspect of the present disclosure.

FIG. 2 is a perspective view of the ingestible capsule of FIG. 1 without the lid to illustrate the interior of the chamber and the microbag therein of the ingestible capsule.

FIG. 3 is a perspective view of the ingestible capsule of FIG. 1 without the lid to illustrate the interior of the chamber and the therapeutic agent therein of the ingestible capsule.

FIG. 4 is a schematic illustration of the ingestible capsule of FIG. 1 depicting release of a therapeutic agent from the chamber of the ingestible capsule upon expansion of the microbag and dislodgement of the lid.

FIG. 5 is a block and schematic diagram illustrating the components of an ingestible capsule according to an aspect of the present disclosure.

FIG. 6 is a block diagram depicting steps that a controller of the ingestible capsule is programmed to perform according to an aspect of the present disclosure.

FIG. 7 is perspective view of an ingestible capsule according to an aspect of the present disclosure.

FIG. 8 is a side partial cut-away view of a first portion of the ingestible capsule of FIG. 7 illustrating the interior of the chamber and microbag therein of the ingestible capsule.

FIG. 9 is a side partial cut-away view of a first portion of the ingestible capsule of FIG. 7 illustrating the interior of the chamber and the therapeutic agent therein of the ingestible capsule.

FIG. 10 is a schematic illustration of the ingestible capsule of FIG. 7 depicting release of a therapeutic agent from the chamber of the ingestible capsule upon expansion of the microbag and dislodgement of the lid.

FIG. 11 is a block diagram of a multi-stage analysis algorithm used to control release of a therapeutic agent according to an aspect of the present disclosure.

FIG. 12 is perspective view of an ingestible capsule according to an aspect of the present disclosure.

FIG. 13 is a side partial cut-away view of a first portion of the ingestible capsule of FIG. 12 illustrating the interior of the chamber and microbag therein of the ingestible capsule.

FIG. 14 is a schematic illustration of the ingestible capsule of FIG. 12 depicting release of a therapeutic agent from the chamber of the ingestible capsule upon expansion of the microbag and dislodgement of the lid.

FIG. 15 is a schematic illustration of an ingestible capsule according to an aspect of the present disclosure depicting release of a therapeutic agent from the chamber of the ingestible capsule upon expansion of the microbag and unhinging of the lid.

FIG. 16 is a block and schematic diagram illustrating the components of an ingestible capsule according to an aspect of the present disclosure.

FIG. 17 is a side partial cut-away view of a first portion of an ingestible capsule illustrating the interior of the chamber according to an aspect of the present disclosure.

FIG. 18 is schematic illustration of a portion of an ingestible capsule prior to release of therapeutic agent according to an aspect of the present disclosure.

FIG. 19 is a schematic illustration of the portion of the ingestible capsule of FIG. 18 after release of therapeutic agent.

FIG. 20 is a perspective view of an ingestible capsule with gastric retention arms in a non-deployed configuration according to an aspect of the present disclosure.

FIG. 21 is a perspective view of the ingestible capsule of FIG. 20 with the gastric retention arms in a deployed configuration.

### DETAILED DESCRIPTION

As used herein with respect to a described element, the terms “a,” “an,” and “the” include at least one or more of the described elements including combinations thereof unless otherwise indicated. Further, the terms “or” and “and” refer to “and/or” and combinations thereof unless otherwise indicated. By “substantially” is meant that the shape or configuration of the described element need not have the mathematically exact described shape or configuration of the described element but can have a shape or configuration that is recognizable by one skilled in the art as generally or approximately having the described shape or configuration of the described element. A “patient” as described herein includes a mammal, such as a human being. The term “top,” “bottom,” “upper,” “lower,” “above,” “below,” and “around” refer to the position or orientation of the components as depicted in the drawings. The terms “first,” “second,” etc. are used to distinguish one element from another and not used in a quantitative sense unless indicated otherwise. The term “plurality” includes two or more of the described components. In addition, when an element is referred to as being “on,” “attached” to, “connected” to, “coupled” with, “contacting,” in “communication” with, “extending” from etc., another element, it can be directly on, attached to, connected to, coupled with, contacting, in communication with, or extending from the other element or intervening elements may also be present. In contrast, when an element is referred to as being, for example, “directly on,” “directly attached” to, “directly connected” to, “directly coupled” with, “directly contacting,” in “direct communication” with, or “directly extending” from another element, there are no intervening elements present. An element that is disposed “adjacent” to another element may have portions that overlap or underlie the adjacent element. A “controller” as used here can include a single controller or multiple controllers.

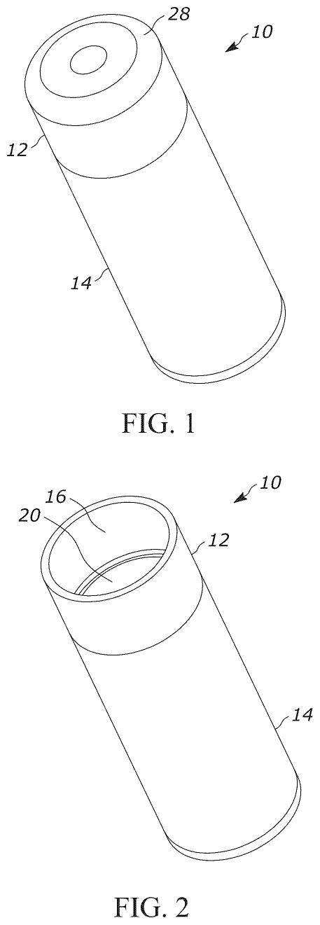

The present disclosure relates to an ingestible capsule that can be configured to release a therapeutic agent(s) within the gastrointestinal tract, such as the stomach, based on electrically triggered commands to improve a medical condition in a patient suffering therefrom. The ingestible capsule can include a single chamber or multiple chambers containing therapeutic agent(s). It should be noted that any suitable aspect or feature of an ingestible capsule with a single chamber can be included in an ingestible capsule with multiple chambers and vice versa. FIGS. 1-5 depicts aspects of an ingestible capsule 10 with a single chamber. Ingestible capsule 10 can include first portion 12 and second portion 14. Chamber 16 can be disposed in first portion 12 and capsule electronics 18 operably coupled to chamber 16 can be disposed in second portion 14.

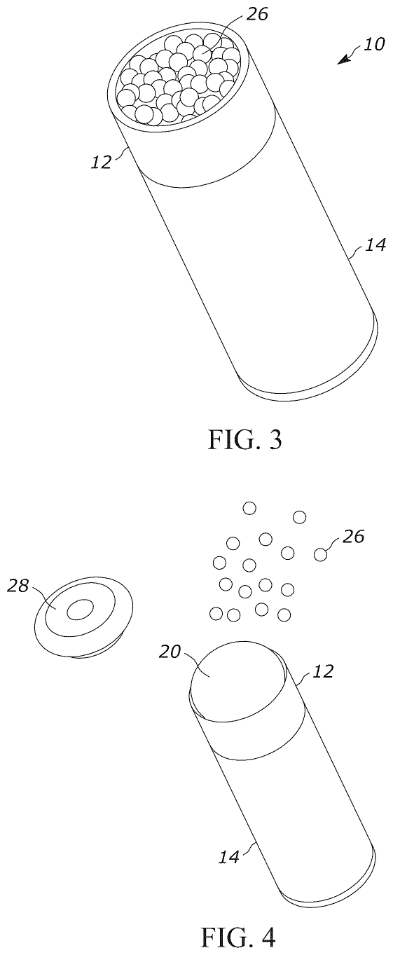

In particular, chamber 16 can comprise microbag 20 that can contain heating filament 22 and chemical substance 24 in communication with heating filament 22 such that a chemical reaction is initiated when the chemical substance is heated by the heating filament to produce a gas. The heating filament can be any suitable electrically driven heating component. For example, the heating filament can be formed of a tightly coiled tungsten wire (or other wire such as, for example, nichrome or an iron compound) or other conductor that achieves a suitable temperature when electric current flows through it. As another example, the heating filament can be a printed, thin film component. Typically, between about 10 milliamperes (mA) and about 250 mA is the preferred amount of current to activate the heating filament. The chemical substance surrounding the heating filament can be any suitable chemical substance that initiates a chemical reaction when heated by the heating filament. For example, the chemical substance can be a mixture of carbon, sulfur, and potassium nitrate or similar chemicals. Typically, between about 0.5 milligrams (mg) and about 2.0 mg of the chemical substance is the preferred amount of chemical substance for the chemical reaction. During the chemical reaction, gases such as carbon dioxide and/or nitrogen can be produced. By including the heating component and chemical substance in a microbag inside a chamber, compounds/substances generated as a result of the chemical reaction can be captured in the bag and not released into the patient's body. The microbag can have an outer surface fabricated of a gas permeable material or membrane (for example, a gas permeable polydimethylsiloxane (PDMS)) such that after inflation and ejection of the therapeutic agent payload (described below), the microbag releases gases generated during the chemical reaction and deflates, while retaining any solid materials from the chemical reaction inside the microbag. Such solid materials would therefore not be released into the patient's body. Chamber 16 can also include therapeutic agent 26 disposed outside of the microbag as depicted in FIG. 3. In particular, the therapeutic agent can be disposed around and/or on top of the microbag. Lid 28 can be attached to chamber 16 and can be configured to releasably retain the therapeutic agent. In other words, the lid can retain the therapeutic agent in the chamber until the lid is commanded to be released as disclosed below.

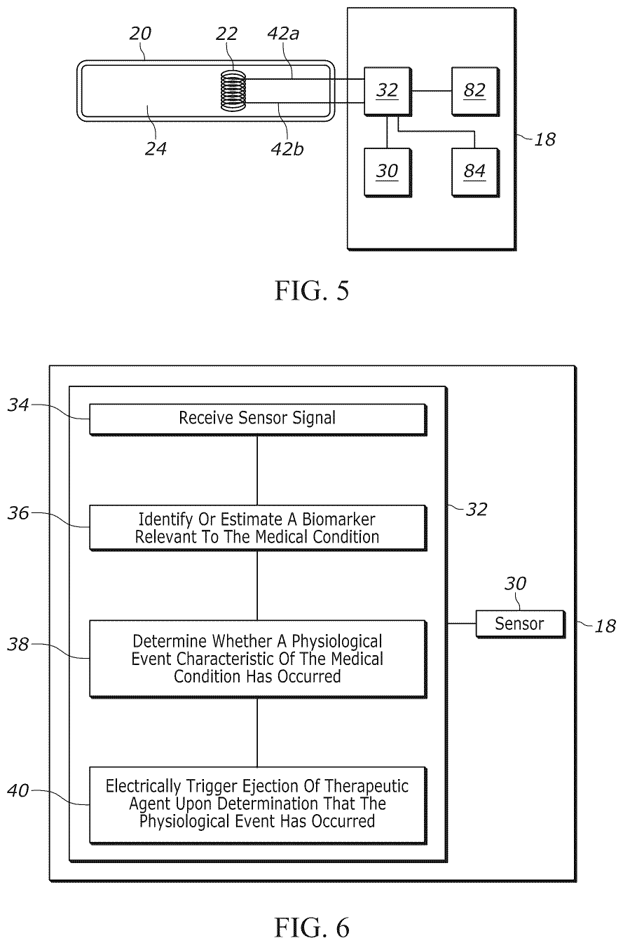

With further reference to FIG. 5, capsule electronics 18 (which can be a printed circuit board (“PCB”)) can include sensor 30 configured to detect a parameter and generate a sensor signal in response to the detection and controller 32 that can receive and process the sensor signal. The sensor can be any suitable sensor that can provide sensed data useful in determining if the therapeutic agent should be released. Likewise, the parameter detected by the sensor can be any suitable parameter useful in making the same determination. The sensor can be, for example, a sensor of accelerometry, body temperature, light, photoplethysmography, voltage, electrical fields, magnetic fields, voltammetry, force, capacitance, pressure, or combinations thereof. In certain aspects, the sensor is an accelerometer including, for example, a sensor of 3-axis accelerometry or 6-axis accelerometry. The parameter detected by the sensor can be, for example, acceleration related to movement of the ingestible capsule within the gastrointestinal tract. With further reference to FIG. 6, controller 32 can be in communication with the sensor and programmed to receive the sensor signal generated by the sensor at step 34 and identify or estimate a biomarker relevant to the medical condition based on the sensor signal at step 36. The biomarker can be any suitable biomarker useful in determining whether the therapeutic agent should be released. For example, the biomarker can be heart rate, R-R interval, heart rate variability, body movement, digestive tract movement, gut motility, body posture, activity level, body temperature, body temperature variation, respiratory rate, respiratory depth, tidal volume, tissue oxygenation, SpO2, pH, presence or absence of respiration, autonomic tone, or combinations thereof. In certain aspects, the biomarker is respiratory rate, heart rate, level of peristaltic activity level, level of overall body movement activity, body temperature, or combinations thereof. At step 38, the controller can determine whether a physiological event characteristic of the medical condition has occurred based on the identification or estimation at step 36. In certain aspects, the controller can determine whether the biomarker falls outside a threshold value or range for the biomarker indicating that a physiological event characteristic of the medical condition has occurred. The physiological event can be any suitable event useful in determining whether the therapeutic agent should be released. For example, the physiological event can be respiratory impairment, body movement impairment, cardiac impairment, an apneic event, or combinations thereof. At step 40, the controller can electrically trigger ejection of the therapeutic agent upon a determination that the physiological event has occurred. In particular, the controller can be programmed to electrically trigger the heating filament to generate heat sufficient to initiate the chemical reaction to produce the gas to inflate the microbag to a sufficient size and configuration to dislodge the lid and thereby eject the therapeutic agent out of the chamber (as schematically illustrated in FIG. 4) into the gastrointestinal tract upon a determination that the physiological event has occurred. The foregoing steps are non-exclusive and the controller can be programmed to perform other additional steps.

Referring back to FIG. 5, controller 32 can be electrically coupled to heating filament 22 via wires/conductors 42. As such, heating filament 22 can be electrically triggered by the controller via a pair of wires/conductors 42a and 42b connecting the chamber's heating filament to the capsule electronics. An adhesive can be used to seal the ends of the wires/conductors in the microbag that are electrically coupled to the heating filament. Controller 32 can be powered by a power source 82 such as internal batteries. The electrical signal used to generate heat in the heating filed can be, for example, programmatically controlled by the controller based on a wireless command signal received by integrated radio module 84 that is communicated to the controller, or based on an algorithm that can use physiological data from sensor 30 to determine that the therapeutic agent(s) should be released when a particular clinical event or biomarker is detected as disclosed above.

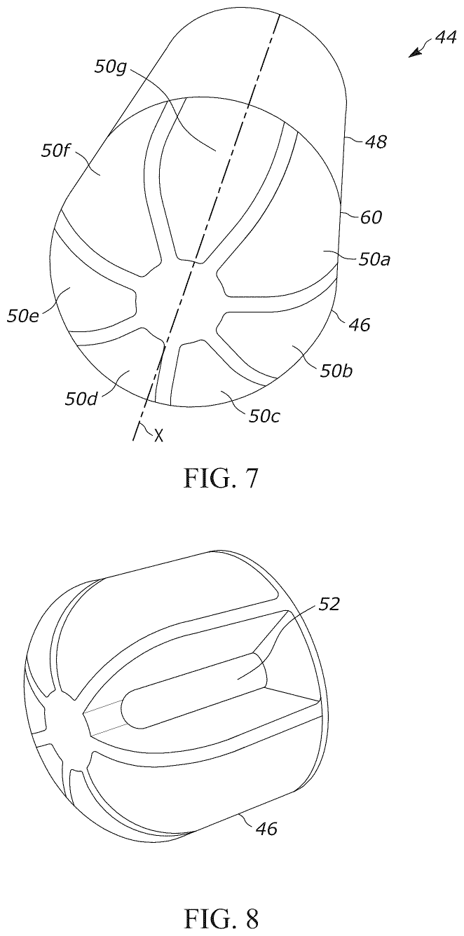

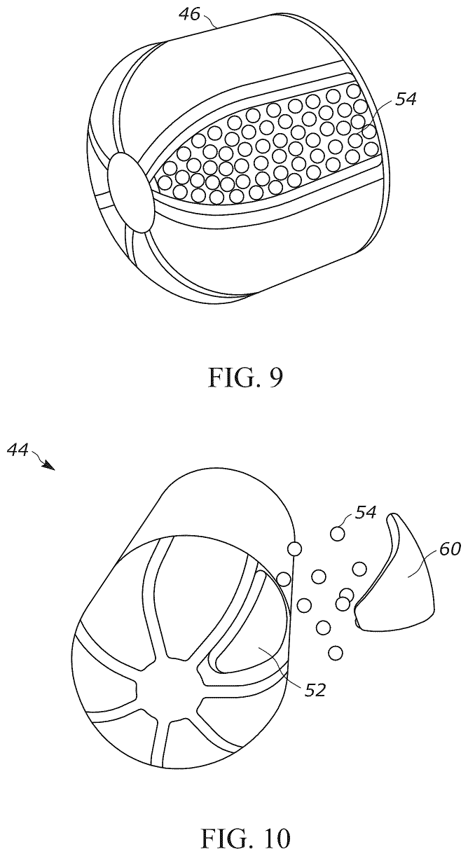

As stated above, an ingestible capsule can include multiple chambers and an embodiment of such a configuration is depicted in FIGS. 7-10. Ingestible capsule 44 has first portion 46, second portion 48, and a longitudinal axis X extending therethrough. A plurality of chambers 50 can be disposed in first portion 46 and radially arranged about the longitudinal axis X of the ingestible capsule. Although FIGS. 7-10 illustrate seven chambers radially arranged about the longitudinal axis of the ingestible capsule, the ingestible capsule can include a different number of chambers and can have a different arrangement of the chambers. The number of chambers can range from one to ten, for example, depending on the size of the desired therapeutic agent(s) payload. Each chamber can be wedge-shaped and also curve inward at the end of the first portion of the ingestible capsule, as best seen in FIG. 8, to maximize volumetric efficiency while maintaining a domed shape end of the capsule housing to maintain the standard form factor of a capsule that is suitable for swallowing. The standard form factor of the ingestible capsule can be 000 to 5.

Similar to the embodiment described above, each of the plurality of chambers 50 can comprise a microbag 52 containing a heating filament and a chemical substance in communication with the heating filament such that a chemical reaction is initiated when the chemical substance is heated by the heating filament to produce a gas. Chamber 50 can also include therapeutic agent 54 disposed outside of the microbag. The ingestible capsule can further include a plurality of lids 60 each attached to a respective one of the plurality of chambers 50 and configured to releasably retain therapeutic agent 54. Second portion 48 of ingestible capsule 44 can include capsule electronics, which can include a sensor and a controller, similar to the sensor and controller disclosed above. In particular, the sensor can be configured to detect a parameter and generate a sensor signal in response to the detection. The controller can be operably coupled to each of the plurality of chambers 50 and can be programmed to perform the steps as disclosed above. In particular, the controller can be programmed to receive the sensor signal; identify or estimate a biomarker relevant to the medical condition based on the sensor signal; determine whether a physiological event characteristic of the medical condition has occurred based on the identification or estimation; and electrically trigger the heating filament to generate heat sufficient to initiate the chemical reaction to produce the gas to inflate the respective microbag to a sufficient size and configuration to dislodge the respective lid and thereby eject the therapeutic agent out of the respective one of the plurality of chambers (as schematically illustrated in FIG. 10) into the gastrointestinal tract upon a determination that the physiological event has occurred. The foregoing steps are non-exclusive and the controller can be programmed to perform other additional steps. The controller can be programmed to release a single therapeutic agent or multiple therapeutic agents, including multiple different types of therapeutic agents. The controller can also be programmed to release the same dose, different doses, or different release times of the therapeutic agent. In certain aspects, the controller can be programmed to independently and selectively release a therapeutic agent from the plurality of chambers. For example, the controller can be programmed to independently and selectively release multiple doses of a therapeutic agent from within the gastrointestinal tract based on the electrically triggered commands. In certain aspects, the controller can be programmed to actuate the release of therapeutic agents from the plurality of chambers based on a specific time schedule.

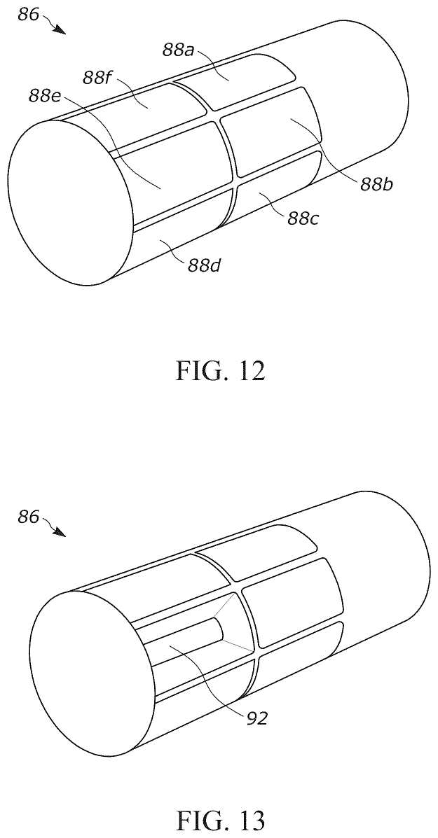

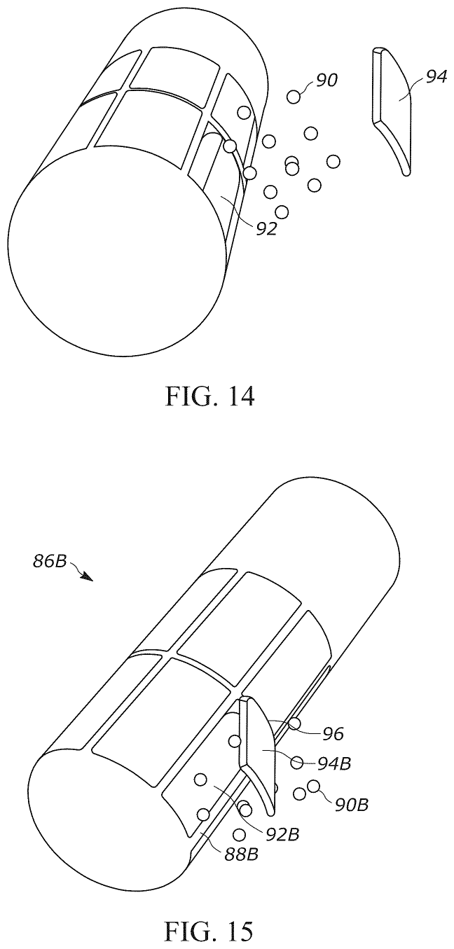

In another embodiment, and with reference to FIGS. 12-15, ingestible capsule 86 can includes stacked sets of radially configured chambers 88 that can provide multiple (e.g. 14) independent doses of therapeutic agent 90. Each chamber can operate in a similar fashion as disclosed above, with a packaged microbag 92 (illustrated in FIG. 13) that inflates (as illustrated in FIG. 14) to detach chamber lid 94 and release therapeutic agent 90 from the chamber. In an embodiment and with reference to FIG. 5, chamber lid 94B can be hinged and remains attached to the outer face defining chamber 88B along an edge 96 of the lid when the microbag 92B expands and therapeutic agent 90B is released. Such a configuration may be advantageous since the lid does not completely detach from the outer surface of the capsule.

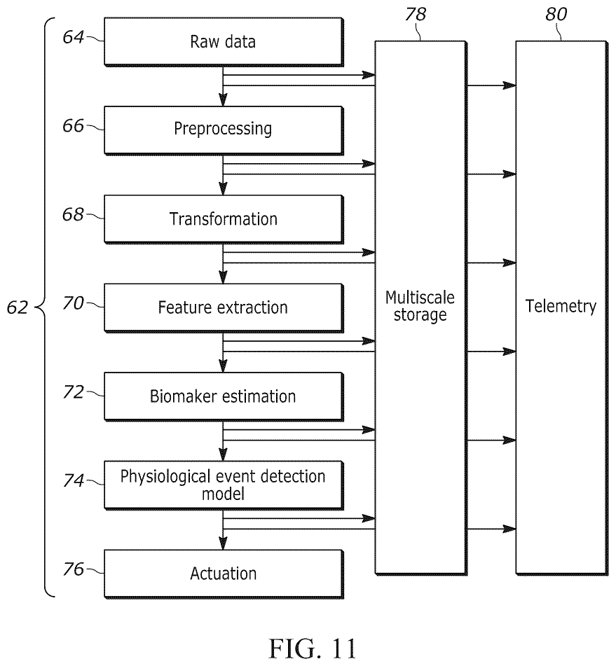

A specific non-limiting implementation of the ingestible capsule, and in particular the steps the controller can be programmed to perform, is disclosed with reference to FIG. 11. The controller can collect raw data (e.g. a parameter) 64 from one or more sensors and use algorithm 62 to trigger release of a therapeutic agent from chamber(s). The raw data collected can be, for example, 3-axis accelerometer data and temperature data. Data passed between steps of algorithm 62 can include multiple channels or multivariate data.

Sensor data can be filtered and/or preprocessed in block 66, for instance by lowpass filtering of accelerometer data to isolate likely respiratory or cardiac ranges, or to allow assessment of movement states. Accelerometer data can be filtered, for example, with a filter such as a 4^{th}-order Butterworth filter, for instance without limitation a bandpass filter with passband 0.1 Hz to 0.5 Hz to accentuate respiratory activity, a bandpass filter with passband 0.67 Hz to 3.33 Hz to accentuate cardiac activity, a bandpass filter with passband 0.1 Hz to 3.33 Hz to accentuate both respiratory and cardiac activity, a low-pass filter with cutoff 0.067 Hz to accentuate peristaltic activity, a bandpass filter with passband 0.067 Hz to 3.33 Hz to accentuate broad-spectrum movement events, or combinations or variations thereof. Preprocessing in block 66 can also include downsampling, such as downsampling from 25 Hz to 12.5 Hz, including downsampling after use of a low-pass filter, in order to save memory and processing time in later steps.

Preprocessed and/or raw sensor data can be transformed in block 68 to yield transformed data suitable for feature extraction. Transformations applied in block 68 can include a combination of one or more of the following, without limitation: Fourier transform, Hilbert transform, wavelet transform, empirical mode decomposition, autoregressive spectrum determination, principal component analysis, or independent component analysis. Preprocessed data in blocks of 0.5 sec duration can be, for example, Hann-windowed and subjected to a fast Fourier transform (FFT), and FFT results for 30 seconds of data, for example, can be stored in a rolling buffer such that averaging across the buffer yields a constantly-updated estimate of the periodogram.

Transformed data can be analyzed in block 70 to extract features suitable for physiological event detection. Preprocessed and/or raw sensor data, for example, can be alternatively or additionally passed directly to block 70 for feature extraction. Features extracted can be, for example, location and prominence (i.e., difference between the peak and its lowest adjacent valley) of spectral peaks, a flag for each peak indicating whether it was observed on multiple channels, and a flag for each peak indicating whether it was observed on the channel with the greatest total power. Features extracted can include one or more of the following, or combinations thereof, without limitation: zero-crossings; power spectrum; power in a band; spectral peak location; spectral peak width; spectral peak prominence; total power in a channel; consistency of spectral peaks across multiple channels; consistency of spectral peaks with peaks estimated in previous data; presence of inter-peak harmonic frequency relationships; power spectrum normalized to account for 1/f, 1/f², or similar falloff profiles; alignment between spectral peak locations and a priori expected frequency bands; or combinations thereof.

Features extracted in block 70 can be passed to one or more biomarker estimation algorithms in block 72; in some aspects, data from previous steps can be directly passed to block 72. Biomarkers estimated may include, without limitation: respiratory rate, respiratory amplitude, heart rate, tidal volume, peristaltic activity level, whole-body activity level, body temperature, and autonomic tone. In certain aspects, five biomarkers can be estimated in block 72: respiratory rate, by selecting the frequency of the most prominent peak in the respiratory frequency band which occurs in two or more channels simultaneously, after normalization for a 1/f falloff profile; heart rate, by selecting the frequency of the most prominent peak in the cardiac frequency band that occurs in two or more channels simultaneously and is not aligned with a harmonic of the respiratory peak; level of peristaltic activity by selecting total power across channels in the peristaltic frequency band; level of overall movement activity by selecting total broad-spectrum power across channels; and body temperature.

Biomarker estimations can be further subjected to smoothing or additional analyses; additionally or alternatively, biomarkers can be estimated from features and other data in block 72 by means such as, for example, linear models, support vector machines, neural networks, convolutional neural networks, forest classifiers, decision tree models, or similar methods. In certain aspects, respiratory rate and heart rate can be estimated using a neural network model whose input is the power spectrum of accelerometer data and/or related features extracted in block 70, and which has been trained on individual data or group data to provide respiratory and heart rate estimates in alignment with measurements made using conventional methods such as polysomnography. In another aspect, respiratory rate and heart rate can be estimated by first using a decision tree model to generate a quality score for each peak identified in the power spectrum, using features extracted in block 70, after which the respiratory rate and heart rate can be determined from the peaks with the highest quality score in the respiratory and cardiac bands, respectively.

Biomarkers estimated in block 72 can be passed to a physiological event detection model in block 74; additionally or alternatively, physiological event detection can also use data directly passed by previous blocks. Physiological events detected can include, without limitation: respiratory impairment including lack of respiration, body movement impairment including lack of body movement, cardiac impairment, apneic events, or combinations thereof. Physiological event detection can include, for example, rule-based detection of respiratory impairment. For instance, a respiratory impairment event can be detected if the respiratory rate estimated in block 72 falls below a threshold, such as 4 breaths per minute, for more than five consecutive minutes, while body movement is negligible. Additionally or alternatively, physiological event detection can occur in block 74 by means such as, for example, linear models, support vector machines, neural networks, convolutional neural networks, forest classifiers, decision tree models, or similar methods. Physiological event detection can trigger actuation of a therapeutic agent at block 76; for instance, detection of sustained respiratory impairment can trigger release of a therapeutic agent.

In embodiments of an ingestible capsule having a plurality of chambers, physiological event detection occurring in block 74 can trigger multiple releases of therapeutic agent. For example, detection of respiratory impairment can trigger a first release of therapeutic agent, after which the algorithm 62 continues to run, and subsequent re-detection of respiratory impairment can trigger additional releases of therapeutic agent.

The output of one or more stages of algorithm 62 can be stored, for example, in multiscale storage 78, implemented by means such as FRAM, flash memory, dynamic RAM, or EEPROM. Multiscale storage 78 can enable efficient use of memory; for instance, available memory can concurrently hold one hour of raw data in a first rolling buffer, one day of transformed data in a second rolling buffer, and a complete record of features extracted, biomarker estimates, physiological event detection activity, and therapeutic agent actuation spanning a one-month device lifetime in a third rolling buffer.

The output of one or more stages of algorithm 62 can made available, for example, to an external host via a wireless telemetry protocol 80, implemented by communication between controller and an integrated radio module. Telemetry 80 can include, for example, real-time streaming of data or streaming of data stored in multiscale storage 78. The controller can also receive commands from the host, such as commands to modify parameters, models, thresholds, or parts of algorithms used in algorithm 62; commands to deliver therapeutic agent; or commands to transfer raw sensor data, compressed biomarker data, or physiological event detection data to the host. The external host can be a device such as, without limitation, a smartphone, computer, tablet, or smart watch. The external host can also use data acquired via telemetry 80 to perform, for example, training or optimization of models used in algorithm 62. For instance, the external host can train a neural network to estimate heart rate using feature extraction data from block 70 and a separate, externally-worn device such as a Holter monitor, after which the trained neural network weights can be transmitted to the controller and the behavior of block 72 in algorithm 62 can be updated. In another example, the external host can identify univariate or multivariate wavelets or empirical modes describing heartbeats or breaths using data from block 66, after which the information describing the wavelet or empirical mode can be transmitted to the controller and the behavior of block 68 in algorithm 62 can be updated.

In alternative embodiments, algorithm 62 can be implemented for monitoring, detection, or diagnostic purposes in an ingestible capsule not having a drug delivery mechanism, or can be used to add monitoring, detection, or diagnostic capabilities to an ingestible capsule having a drug delivery mechanism.

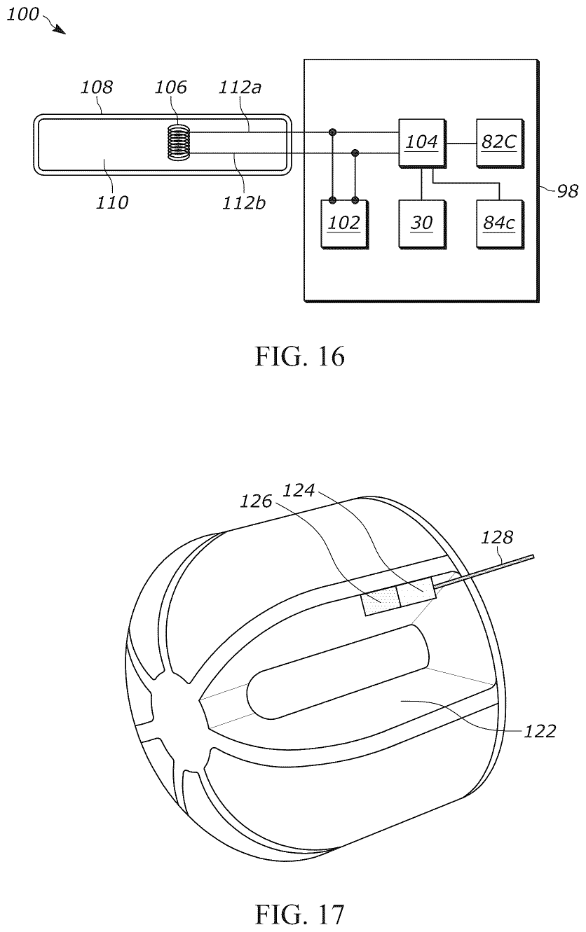

Embodiments disclosed above generally relate to an ingestible capsule where sensed data obtained from a sensor(s) can be processed by a controller to determine when to release a therapeutic agent(s) to improve a medical condition. In certain aspects, the capsule electronics can include a component to confirm that the therapeutic agent has been released and, if not, re-command release of the therapeutic agent(s). For example and with reference to FIG. 16, capsule electronics 98 of ingestible capsule 100 can include an impedance checking circuit including an impedance sensor 102 that is operably coupled to controller 104 and that is configured to measure the impedance of heating filament 106 and generate an impedance signal based on the measurement. Controller 104 can be programmed to receive the impedance signal to determine if the impedance value is below a pre-determined threshold level or above a pre-determined threshold level to determine if the microbag has inflated and therefore the therapeutic agent has been ejected from microbag 108. The controller can be programmed to re-command release of the therapeutic agent upon a determination that the impedance value is below the pre-determined threshold value. In particular, as heating filament 106 initiates the chemical reaction (when chemical substance 110 is heated by the heating filament to produce a gas), resulting in the expansion of microbag 108 and release of the therapeutic agent, heating filament 106 can be broken by the force of the reaction, resulting in an open circuit across the heating filament electrical connections 112a and 112b. Impedance sensor 102 can measure the electrical resistance of the heating filament and when the heating filament is intact, before a command has been received to release the therapeutic agent, the resistance of the heating filament can be measured and if below a pre-determined threshold (e.g. 20 Ohms), deemed intact by the impedance sensor. After the microbag is triggered to release the therapeutic agent, and the heating filament has been broken, the electrical resistance can be measured at a value above a pre-determined threshold (e.g. >100 KOhms). Having the ability to use a sensor to confirm actuation of the microbag (and therefore therapeutic agent release) can provide an additional safety and performance capability for the drug delivery system.

In an embodiment, and with reference to FIG. 17, an ingestible capsule can include chamber 122 containing an optical sensor comprising an LED 124 and photodiode 126 electrically connected to an electrical cable 128 that can interface with the capsule electronics of the ingestible capsule and specifically the controller. The optical sensor can be used to detect when therapeutic agent has released from the chamber by measuring changes in the intensity of light received/sensed by photodiode 126 as the LED 124 emits light. The controller can be programmed to: a) command the photodiode to sense the light intensity in the chamber; b) command release of the therapeutic agent from the chamber; c) re-command the photodiode to re-sense the light intensity in the chamber; and d) re-command release of the therapeutic agent from the chamber upon a determination that the light intensity measurement from step a) is substantially the same as the light intensity measurement from step c). In particular, when the lid covering the chamber is intact, attached to the respective chamber, and therapeutic agent is in the chamber, the photodiode can measure a particular voltage related to light intensity. After the therapeutic agent has been released and the lid has opened/detached from chamber, the voltage from the photodiode 126 proportional to the reflected light intensity from the LED 124 can change by an amount above a pre-determined threshold. The command to sense the reflected light can be issued by the controller, and the photodiode voltage can be measured by the controller. After a specific interval after a therapeutic agent release command has been issued by the controller, the controller can confirm if the therapeutic agent has in fact been released by using the readings from the optical sensor. If the optical sensor does not detect a change, implying the lid did not open and the therapeutic agent did not release, another attempt at releasing the therapeutic agent can be commanded. Similar to the sensor described in FIG. 16, having an optical sensor as shown in FIG. 17 to confirm the therapeutic agent has been released when commanded can provide additional safety and performance capability for the drug delivery system. Other sensors can also be used, for example but not limited to temperature sensors, thermal mass flowmetry sensors, or capacitance sensors.

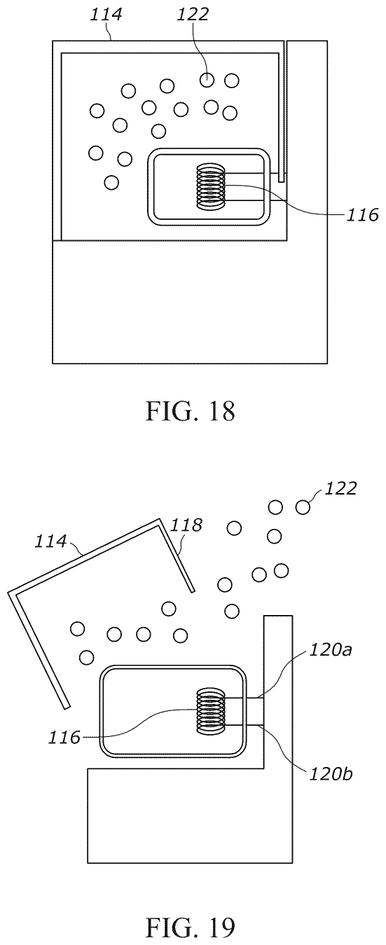

Referring to FIGS. 18 and 19, in an embodiment, a structure may be incorporated into lid 114 of an ingestible capsule and attached onto or near heating filament 116, for instance protrusion 118 of lid 114 which protrudes inward toward heating filament 116 or its electrical connections 120a and 120b and is molded around or hooked around this structure, such that electrical conductivity through heating filament 116 is broken when the lid has sufficiently opened to deliver therapeutic agent 122. This capability can provide additional certainty that therapeutic agent has been released, as well as augmenting the ability of an electrical sensor to detect or confirm release without requiring additional conductors to be routed from the capsule electronics to the first portion of the ingestible capsule.

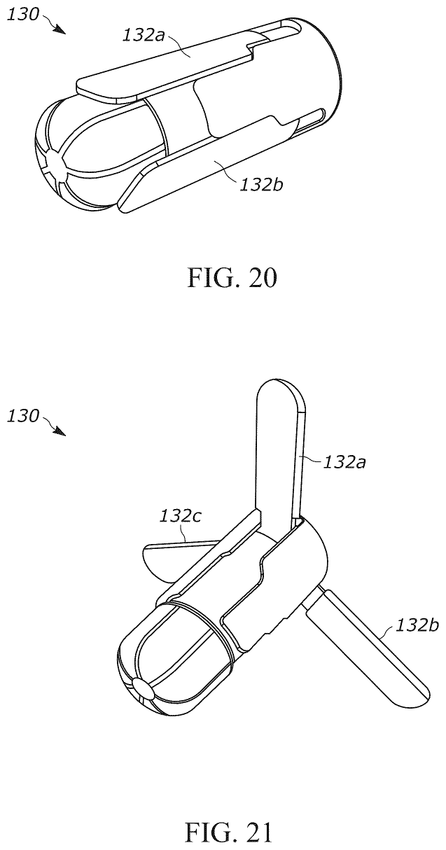

In an embodiment, and with reference to FIGS. 20-21, ingestible capsule 130 can also include gastric retention arms 132 as described in U.S. Provisional Application No. 63/327,108, filed on Apr. 4, 2022 and which is incorporated by reference in its entirety. FIG. 20 illustrates the configuration of the ingestible capsule when the gastric retention arms are non-deployed and FIG. 21 illustrates the configuration of the ingestible capsule when the gastric retention arms are deployed.

Ingestible capsules disclosed herein can be applied to a variety of indications in which it is desirable to deliver, for example, single doses of a therapeutic agent or multiple doses of a therapeutic agent at specific times, based on, for example, input from the user or based on detection of clinically relevant events that requires intervention. Table I provides non-limiting indications and treatments for an ingestible capsule as disclosed herein.

TABLE I

| | Drug(s) Delivered |
|---|---|
| | Singly or in combinations |
| Indication | Autonomously, on-demand, or timed delivery |
| Opioid Overdose Prevention | Opioid Antagonists and Opioid Agonists |
| | Methadone, Naltrexone, Buprenorphine, |
| | Buprenorphine/Naloxone, Lofexidine |
| | Anti-withdrawal medications |
| | Anti-nausea medications |
| Paroxysmal Atrial Fibrillation | Anticoagulant |
| | Antiarrhythmic |
| | Cardiac glycoside (Digoxin) |
| COPD | Anticholinergics |
| | Corticosteroids |
| | Long & Short Acting Beta Agonists |
| | Oral PDE4 inhibitors |
| Asthma | Corticosteroids |
| | Leukotriene modifiers |
| | Long-acting beta agonists (LABAs) |
| | Long-acting muscarinic antagonists (LAMAs) |
| | Theophylline |
| Anxiety/Panic Attacks | Selective serotonin reuptake inhibitors (SSRIs) |
| | Serotonin and norepinephrine reuptake inhibitors |
| | Benzodiazepines |
| | Monoamine oxidase inhibitors (MAOIs) |
| | Atypical antidepressants |
| | Tricyclic antidepressants |
| | Anti-anxiety drugs |
| | Anticonvulsants |
| | Antihypertensives |
| Heart Failure | Cardiac glycoside (Digoxin) |
| Hypothyroidism | Levothyroxine Sodium |
| Hypertension | Antihypertensives |
| Insomnia | Benzodiazepines |
| Seizures | Anticonvulsants |
| | Benzodiazepines |
| Parkinson's | dopamine agonists |
| Schizophrenia | Antipsychotic |
| Birth Control | Combination birth control - both estrogen and progestin |
| | The minipill - only progestin. |
| Cytokine Storm | Cytokine inhibitors |
| | Anti-inflammatory drugs |
| | interleukin-6 (IL-6) receptor inhibitors |
| Organ rejection | Anti-rejection drugs |
| Autoimmune disorders | anti-inflammatory drugs |
| | corticosteroids |
| | pain-killing medication |
| | immunosuppressant drugs |
| | immunosuppressants |
| Food Allergies | Epinephrine |
| | Antihistamines |
| | Corticosteroids |
| Organophosphate nerve agent | atropine |
| poisoning/chemical warfare | pralidoxime |
| | diazepam |
| | benzodiazepines |

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. An ingestible capsule to improve a medical condition in a patient suffering therefrom, the ingestible capsule having a first portion and a second portion and comprising:
a chamber disposed in the first portion comprising:
a microbag having an outer surface and comprising a heating filament and a chemical substance in communication with the heating filament such that a chemical reaction is initiated when the chemical substance is heated by the heating filament to produce a gas; and
a therapeutic agent disposed outside of the microbag;
a lid attached to the chamber and configured to releasably retain the therapeutic agent; and
capsule electronics disposed in the second portion comprising:
a sensor configured to detect a parameter and generate a sensor signal in response to the detection; and
a controller operably coupled to the chamber and programmed to:
receive the sensor signal;
identify or estimate a biomarker relevant to the medical condition based on the sensor signal;
determine whether a physiological event characteristic of the medical condition has occurred based on the identification or estimation; and
electrically trigger the heating filament to generate heat sufficient to initiate the chemical reaction to produce the gas to inflate the microbag to a sufficient size and configuration to dislodge the lid and thereby eject the therapeutic agent out of the chamber into the gastrointestinal tract upon a determination that the event has occurred.

2. The ingestible capsule of claim 1, wherein the sensor comprises a sensor of accelerometry, body temperature, light, photoplethysmography, voltage, electrical fields, magnetic fields, voltammetry, force, capacitance, pressure, or combinations thereof.

3. The ingestible capsule of claim 1, wherein the sensor comprises a sensor of accelerometry.

4. The ingestible capsule of claim 3, wherein the sensor of accelerometry comprises a sensor of 3-axis accelerometry or 6-axis accelerometry.

5. The ingestible capsule of claim 1, wherein the parameter is acceleration related to movement of the capsule within gastrointestinal tract.

6. The ingestible capsule of claim 1, wherein the biomarker is heart rate, R-R interval, heart rate variability, body movement, digestive tract movement, gut motility, body posture, activity level, sleep stage, body temperature, body temperature variation, respiratory rate, respiratory depth, tidal volume, tissue oxygenation, SpO2, pH, presence or absence of respiration, autonomic tone, or combinations thereof.

7. The ingestible capsule of claim 1, wherein the biomarker is respiratory rate, heart rate, level of peristaltic activity, level of overall body movement activity, body temperature, or combinations thereof.

8. The ingestible capsule of claim 1, wherein the physiological event comprises respiratory impairment, body movement impairment, cardiac impairment, an apneic event, or combinations thereof.

9. The ingestible capsule of claim 1, wherein the controller is programmed to determine whether the biomarker falls outside a threshold value or range for the biomarker indicating that a physiological event characteristic of the medical condition has occurred

10. The ingestible capsule of claim 1, wherein at least a portion of the outer surface of the microbag comprises a gas-permeable material or membrane.

11. The ingestible capsule of claim 1, further comprising an impedance checking circuit operably coupled to the controller and configured to measure the impedance of the heating filament and generate an impedance signal based on the measurement.

12. The ingestible capsule of claim 11, wherein the controller is programmed to receive the impedance signal to determine if the impedance value is below a pre-determined threshold level or above a pre-determined threshold level to determine if the microbag has inflated and therefore the therapeutic agent has been ejected from the microbag.

13. The ingestible capsule of claim 12, wherein the controller is programmed to re-command release of the therapeutic agent upon a determination that the impedance value is below the pre-determined threshold value.

14. The ingestible capsule of claim 1, further comprises a photodiode and a light emitting diode (LED), the photodiode configured to sense an intensity of light received by the photodiode as the LED emits light.

15. The ingestible capsule of claim 14, wherein the controller is programmed to:
a) command the photodiode to sense the light intensity in the chamber;
b) command release of the therapeutic agent from the chamber;
c) re-command the photodiode to re-sense the light intensity in the chamber; and
d) re-command release of the therapeutic agent from the chamber upon a determination that the light intensity measurement from step a) is the substantially the same as the light intensity measurement from step c).

16. An ingestible capsule to improve a medical condition in a patient suffering therefrom, the ingestible capsule having a first portion, a second portion, and a longitudinal axis extending therethrough, the ingestible capsule comprising:
a plurality of chambers disposed in the first portion and radially arranged about the longitudinal axis of the ingestible capsule, each of the plurality of chambers comprising:
a microbag having an outer surface and comprising a heating filament and a chemical substance in communication with the heating filament such that a chemical reaction is initiated when the chemical substance is heated by the heating filament to produce a gas; and
a therapeutic agent disposed outside of the microbag;
a plurality of lids, each attached to a respective one of the plurality of chambers and configured to releasably retain the therapeutic agent; and
capsule electronics disposed in the second portion comprising:
a sensor configured to detect a parameter and generate a sensor signal in response to the detection; and
a controller operably coupled to each of the plurality of chambers and programmed to:
receive the sensor signal;
identify or estimate a biomarker relevant to the medical condition based on the sensor signal;
determine whether a physiological event characteristic of the medical condition has occurred based on the identification or estimation; and
electrically trigger the heating filament to generate heat sufficient to initiate the chemical reaction to produce the gas to inflate the respective microbag to a sufficient size and configuration to dislodge the respective lid and thereby eject the therapeutic agent out of the respective one of the plurality of chambers into the gastrointestinal tract upon a determination that the event has occurred.

17. The ingestible capsule of claim 16, wherein the controller is programmed to command independent and selective release of the therapeutic agent from each of the plurality of chambers.

18. The ingestible capsule of claim 16, wherein the dose and/or the type of the therapeutic agent in at least one of the plurality of chambers is different from the dose and/or type of the therapeutic agent in at least another one of the plurality of chambers.

19. The ingestible capsule of claim 16, wherein the controller is programmed to control release of the therapeutic agent from at least one of the plurality of chambers at a different time than release of the therapeutic agent from at least another one of the plurality of chambers.

20. The ingestible capsule of claim 16, wherein the controller is programmed to actuate release of the therapeutic agent from the plurality of chambers based on a specific time schedule.

21. The ingestible capsule of claim 16, wherein at least a portion of the outer surface of the bag comprises a gas-permeable material or membrane.

22. The ingestible capsule of claim 16, wherein each of the plurality of lids is hingedly attached to a respective one of the plurality of chambers and configured to releasably retain the therapeutic agent.
